(19) United States
(12) Patent Application Publication
(10) Pub. No.: US 2022/0257603 A1
(43) Pub. Date: Aug. 18, 2022

(54) COMBINATION THERAPY FOR TREATING MTAP-DEFICIENT TUMORS

(71) Applicant: Board of Regents, The University of Texas System, Austin, TX (US)

(72) Inventor: Jianjun GAO, Houston, TX (US)

(73) Assignee: Board of Regents, The University of Texas System, Austin, TX (US)

(21) Appl. No.: 17/618,778

(22) PCT Filed: Jun. 15, 2020
(86) PCT No.: PCT/US2020/037712

§ 371 Date: Dec. 13, 2021

Related U.S. Application Data
(60) Provisional application No. 62/860,963, filed on Jun. 13, 2019.

(51) Int. Cl. A61K 31/522; A61K 31/519; A61K 31/513; A61K 31/155; A61K 31/505; A61K 39/395; A61K 45/06; A61P 35/00

(52) U.S. Cl. CPC A61K 31/522; A61K 31/519; A61K 31/513; A61P 35/00; A61K 31/505; A61K 39/3955; A61K 45/06; A61K 31/155

(57) ABSTRACT

Provided herein are methods of treating patients with a combination of an anti-folate agent and an A2BR antagonist. Also provided are methods of treating patients with a combination of an anti-folate agent and an immune checkpoint inhibitor. The patients are selected for treatment based on having an MTAP-deficient cancer.

### REFERENCE TO RELATED APPLICATIONS

The present application claims the priority benefit of U.S. provisional application No. 62/860,963, filed Jun. 13, 2019, the entire contents of which is incorporated herein by reference.

### STATEMENT REGARDING FEDERALLY SPONSORED RESEARCH

This invention was made with government support under grant number CA091846 awarded by the National Institutes of Health. The government has certain rights in the invention.

### BACKGROUND

### 1. Field

The present invention relates generally to the field of oncology. More particularly, it concerns methods of selecting patients having MTAP-deficient cancers for treatment with a combination of an anti-folate agent and an A2BR antagonist and/or an immune checkpoint inhibitor as well as methods of treating patients so selected.

### 2. Description of Related Art

Homozygous genetic deletion at chromosome 9p21 of methylthioadenosine phosphorylase (MTAP) is a common event observed in various cancer types. MTAP degrades methylthioadenosine (MTA), a byproduct of polyamine synthesis, into methylthioribose-1′-phosphate (MTR-1'-P) and adenine, which are recycled into the methionine and purine salvage pathways. MTAP loss is correlated with a poor prognosis. Deletion or repression of MTAP leads to the buildup and excretion of MTA, which was recently shown to have potent immunosuppressive properties. Incubation with MTA halts the proliferation and differentiation of naive lymphocytes and is cytotoxic to activated human T cells. Tumor excreted MTA may be considered an immune checkpoint that helps tumor cells evade immune surveillance and elimination. As such, methods for treating MTAP-deficient cancers, including methods to overcoming resistance to immune checkpoint inhibitor therapy, are needed.

### SUMMARY

In one embodiment, provided herein are methods of treating a patient having a cancer, the methods comprising (a) determining or having determined the level of MTAP protein expression in the patient's cancer; (b) selecting or having selected the patient for treatment with an anti-folate agent and an A2BR antagonist when the patient's cancer has a decreased level of MTAP expression relative to a level of MTAP protein expression in a reference sample; and (c) administering or having administered to the selected patient a combined therapeutically effective amount of an anti-folate agent and an A2BR antagonist. In some aspects, step (a) comprises (i) obtaining or having obtained a biological sample from the patient; and (ii) performing or having performed an assay on the biological sample to determine the level of MTAP protein expression in the patient's cancer. In some aspects, the methods further comprise administering to the patient an immune checkpoint inhibitor.

In one embodiment, provided herein are methods of treating a patient having a cancer, the methods comprising administering to the patient a combined therapeutically effective amount of an anti-folate agent and an A2BR antagonist, wherein the cancer has a decreased level of MTAP expression relative to a level of MTAP protein expression in a reference sample. In some aspects, the methods further comprise administering to the patient an immune checkpoint inhibitor.

In one embodiment, provided herein are methods of selecting a patient having a cancer for treatment with an anti-folate agent and an A2BR antagonist, the methods comprising (a) determining or having determined the level of MTAP protein expression in the patient's cancer; and (b) selecting or having selected the patient for treatment with an anti-folate agent and an A2BR antagonist when the patient's cancer has a decreased level of MTAP expression relative to a level of MTAP protein expression in a reference sample. In some aspects, step (a) comprises (i) obtaining or having obtained a biological sample from the patient; and (ii) performing or having performed an assay on the biological sample to determine the level of MTAP protein expression in the patient's cancer. In some aspects, the methods further comprise (c) administering or having administered to the selected patient combined therapeutically effective amounts of an anti-folate agent and an A2BR antagonist.

In one embodiment, provided herein are methods of treating a patient having a cancer, the methods comprising (a) determining or having determined the level of MTAP protein expression in the patient's cancer; (b) selecting or having selected the patient for treatment with an anti-folate agent and an immune checkpoint inhibitor when the patient's cancer has a decreased level of MTAP expression relative to a level of MTAP protein expression in a reference sample; and (c) administering or having administered to the selected patient a combined therapeutically effective amount of an anti-folate agent and an immune checkpoint inhibitor. In some aspects, step (a) comprises (i) obtaining or having obtained a biological sample from the patient; and (ii) performing or having performed an assay on the biological sample to determine the level of MTAP protein expression in the patient's cancer. In some aspects, the methods further comprise administering to the patient an A2BR antagonist.

In one embodiment, provided herein are methods of treating a patient having a cancer, the methods comprising administering to the patient a combined therapeutically effective amount of an anti-folate agent and an immune checkpoint inhibitor, wherein the cancer has a decreased level of MTAP expression relative to a level of MTAP protein expression in a reference sample. In some aspects, the methods further comprise administering to the patient an A2BR antagonist.

In one embodiment, provided herein are methods of selecting a patient having a cancer for treatment with an anti-folate agent and an immune checkpoint inhibitor, the method comprising (a) determining or having determined the level of MTAP protein expression in the patient's cancer; and (b) selecting or having selected the patient for treatment with an anti-folate agent and an immune checkpoint inhibitor when the patient's cancer has a decreased level of MTAP expression relative to a level of MTAP protein expression in a reference sample. In some aspects, step (a) comprises (i) obtaining or having obtained a biological sample from the patient; and (ii) performing or having performed an assay on the biological sample to determine the level of MTAP protein expression in the patient's cancer. In some aspects, the methods further comprise (c) administering or having administered to the selected patient combined therapeutically effective amounts of an anti-folate agent and an immune checkpoint inhibitor. In some aspects, the methods further comprise administering to the patient an A2BR antagonist.

In some aspects of any of the present embodiments, the anti-folate agent comprises pemetrexed, methotrexate, trimetrexate, edatrexate, lometrexol, 5-fluorouracil, pralatrexate, aminopterin, proguanil, pyrimethamine, or trimethoprin. In some aspects of any of the present embodiments, the A2BR antagonist comprises MRS1706, MRS1754, PSB603, CVT-6883, or PBF-1129, or an adenosine 2A (A2A) receptor antagonist with overlapping inhibiting activity of A2BR.

In some aspects of any of the present embodiments, the immune checkpoint inhibitor comprises one or more of an anti-PD1 therapy, an anti-PD-L1 therapy, and an anti-CTLA-4 therapy. In some aspects, the anti-PD1 therapy comprises nivolumab, pembrolizumab, pidilizumab, cemiplimab, tislelizumab, spartalizumab, PF-06801591, AK105, BCD-100, BI-754091, HLX10, JS001, LZMO09, MEDI 0680, MGA012, Sym021, TSR-042, MGD013, AK104, and/or XmAb20717. In some aspects of any of the present embodiments, the anti-PD-L1 therapy comprises atezolizumab, avelumab, durvalumab, FS118, BCD-135, BGB-A333, CBT502, CK-301, CS1001, FAZ053, KN035, MDX-1105, MSB2311, SHR-1316, M7824, LY3415244, CA-170, and CX-072. In some aspects of any of the present embodiments, the anti-CTLA-4 therapy comprises ipilimumab, tremelimumab, BMS-986218, AK104, and/or XmAb20717.

In some aspects of any of the present embodiments, the reference sample is obtained from healthy or non-cancerous tissue in the patient. In some aspects, the reference sample is obtained from a healthy subject.

In some aspects of any of the present embodiments, the cancer has an MTAP deletion.

In some aspects of any of the present embodiments, the methods further comprise administering a further anti-cancer therapy to the patient. In some aspects of any of the present embodiments, the further anti-cancer therapy is a surgical therapy, a chemotherapy, a radiation therapy, a cryotherapy, a hormonal therapy, a toxin therapy, an immunotherapy, or a cytokine therapy. In some aspects of any of the present embodiments, the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma. In some aspects of any of the present embodiments, the cancer is a bladder cancer, a lung cancer, a mesothelioma, a glioblastoma, or a lymphoma.

In some aspects of any of the present embodiments, the patient has previously undergone at least one round of anti-cancer therapy. In some aspects of any of the present embodiments, the patient has previously failed to respond to the administration of an immune checkpoint inhibitor. In some aspects, the method is a method of overcoming resistance to immune checkpoint inhibitor therapy. In some aspects of any of the present embodiments, the method is further defined as a method for increasing sensitivity to immunotherapy.

In some aspects of any of the present embodiments, the methods further comprise reporting the level of MTAP protein expression in the patient's cancer. In some aspects, reporting comprises preparing a written or electronic report. In some aspects of any of the present embodiments, the methods further comprise providing the report to the subject, a doctor, a hospital, or an insurance company.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. (FIG. 1A) MTAP homozygous (HD) deletion rate in TCGA. (FIG. 1B) MTAP protein deficiency rate per IHC of BC TMA. (FIG. 1C) Median overall survival (OS) of MTAP-wild type vs MTAP-deficient bladder cancer patients in TCGA. (FIG. 1D) Median OS of MTAP-wild type vs MTAP-deficient bladder cancer patients in MD Anderson database.

FIGS. 2A-H. (FIG. 2A) Upper panel shows the percentage of sub-G1 population (i.e., apoptotic) MTAP-deficient vs. MTAP-wild type human bladder cancer cells following treatment with pemetrexed. Lower panel shows a western blot of MTAP corresponding to the 8 cell lines in the upper panel. (FIG. 2B) Graph shows the percentage of sub-G1 population (i.e., apoptotic) following knockdown of MTAP in human bladder cancer cell lines. The insert shows a western blot of MTAP. (FIG. 2C) Graph shows the effect of pemetrexed treatment on MTAP-deficient human tumor growth in a xenograft mouse model. (FIG. 2D) Graph shows the effect of pemetrexed treatment on MTAP-wild type tumor growth in a xenograft mouse model. (FIG. 2E) Graph shows the combined effect of MTAP knockdown and pemetrexed treatment on human tumor growth in a xenograft mouse model. (FIG. 2F) Graph shows the best response for target lesions of MTAP-deficient bladder cancer vs MTAP-wild type bladder cancer patients based on a retrospective analysis, based on maximal percentage of tumor reduction per RECIST criteria. (FIG. 2G) Graph shows the best response for target lesions of MTAP-deficient bladder cancer vs MTAP-wild type bladder cancer patients enrolled in a prospective trial, based on maximal percentage of tumor reduction per RECIST criteria. Asterisk represents a patient that died of a car accident and thus was not evaluable. (FIG. 2H) Graph shows the combined retrospective and prospective rate of pemetrexed in patients with MTAP-deficient bladder cancer vs MTAP-wild type bladder cancer.

FIGS. 3A-D. (FIG. 3A) Graph shows the expression levels of PD-L1 in MTAP-deficient human bladder cancer cell lines compared to MTAP-proficient bladder cancer cell lines. (FIG. 3B) TCGA PD-L1 gene expression analysis of MTAP-deficient tumors. (FIG. 3C) TCGA CD274 expression analysis of MTAP-deficient tumors. (FIG. 3D) TCGA resting dendritic cell signature expression analysis of MTAP-deficient tumors. (FIG. 3E) Retrospective data analysis of a patient with metastatic MTAP-deficient bladder cancer that was pre-treated with pemetrexed and then treated with anti-PD1/PD-L1 therapy. Tumors are circled.

FIGS. 4A-C. (FIG. 4A) Graph shows the fold change in PD-L1 expression following treatment with pemetrexed in MTAP-deficient bladder cancer cell lines. (FIG. 4B) Graph shows the percent of immune cells (i.e., CD4/8 T cells, macrophages, dendritic cells, and myeloid-derived suppressor cells) with and without in vivo pemetrexed treatment in vivo in MB49 tumors. (FIG. 4C) Graph shows the expression level of PD-L1 induced by treatment with pemetrexed in vivo in MB49 tumors.

FIGS. 5A-B. (FIG. 5A) Graph shows the effect of MTA on the production of INF-γ. (FIG. 5B) Graph shows the effect of MTA on the production of TNF-α.

FIG. 6. Pemetrexed and Avelumab Combination Phase 2 Trial Schema.

FIGS. 7A-B. (FIG. 7A) Graph shows the effect of MTA, BAY60-6385, and PSB603+MTA on the production of INF-γ. (FIG. 7B) Graph shows the effect of MTA, BAY60-6385, and PSB603+MTA on the production of TNF-α.

### DETAILED DESCRIPTION

Many types of tumors contain homozygous deletion of the methylthioadenosine phosphorylase (MTAP) gene, which encodes an essential enzyme to catalyze methylthioadenosine (MTA) in the salvage pathway for adenosine synthesis. As expected, MTAP loss results in accumulation of its substrate MTA, which acts through its receptor A2BR to inhibit IFN signaling and T cell function. Therefore, MTAP-deficient tumors harbor a relatively “cold” tumor immune microenvironment that is not favorable to immunotherapy. MTAP becomes critical for cell proliferation and survival when the de novo pathway for adenosine synthesis is blocked by anti-folate agents, such as pemetrexed, methotrexate, and pralatrexate. As such, pemetrexed is extremely cytotoxic to MTAP-deficient tumors due to synthetic lethality. In vitro, in vivo, and patient data confirm this hypothesis. In addition, anti-folate agents such as pemetrexed and methotrexate can induce PD-L1 expression on tumor cells both in vitro and in vivo. Furthermore, pemetrexed treatment in vivo can cause increased immune cell infiltration into tumors. Therefore, anti-folate agents can modulate the tumor immune microenvironment for better response to immune checkpoint therapy agents such as anti-PD1 or anti-PD-L 1. Additionally, in vitro data showed that MTA or specific agonist receptor A2BR can inhibit T cell function, whereas A2BR antagonist can reverse inhibition of T cell function by MTA. As such, combination therapy with antifolate agents (e.g., pemetrexed) plus A2BR antagonists are useful for the treatment of MTAP-deficient malignancies. The triple combination of anti-folate agents (e.g., pemetrexed) plus A2BR antagonists plus anti-PD1 or anti-PD-L1 is expected to be an even more effective treatment for MTAP-deficient malignancies.

### I. ANTI-FOLATE AGENTS

Folic acid or folate is a B vitamin. It is also referred to as vitamin M, vitamin B₉, vitamin B_{c} (or folacin), pteroyl-L-glutamic acid, and pteroyl-L-glutamate. Folic acid is synthetically produced, and used in fortified foods and supplements. Folate is converted by humans to dihydrofolate (dihydrofolic acid; DHF), tetrahydrofolate (tetrahydrofolic acid; THF), and other derivatives, which have various biological activities. Vitamin B₉ is essential for numerous bodily functions. Humans cannot synthesize folates de novo; therefore, folic acid has to be supplied through the diet to meet the daily requirements. The human body needs folate to synthesize DNA, repair DNA, and methylate DNA as well as to act as a cofactor in certain biological reactions. It is especially important in aiding rapid cell division and growth, such as in infancy and pregnancy. Children and adults both require folate to produce healthy red blood cells and prevent anemia. Folates occur naturally in many foods and, among plants, are especially plentiful in dark green leafy vegetables. Folate is important for cells and tissues that rapidly divide.

Cancer cells divide rapidly, and drugs that interfere with folate metabolism are used to treat cancer. Antifolates constitute an established class of pharmacological agents that antagonize or block the effects of folic acid on cellular processes. Folic acid's primary function in the body is as a cofactor to various methyltransferases involved in serine, methionine, thymidine and purine biosynthesis. Consequently, antifolates inhibit cell division, DNA/RNA synthesis and repair, and protein synthesis. The majority of antifolates work by inhibiting dihydrofolate reductase (DHFR), and thereby inhibiting the production of the active form of THF from inactive DHF. Antifolates act specifically during DNA and RNA synthesis, and thus are cytotoxic during the S-phase of the cell cycle. Thus, they have a greater toxic effect on rapidly dividing cells (such as malignant and myeloid cells, and GI & oral mucosa), which replicate their DNA more frequently, and thus inhibits the growth and proliferation of these non-cancerous cells as well as causing certain side-effects.

Methotrexate, abbreviated MTX and formerly known as amethopterin, is an antimetabolite and antifolate drug. It is used in treatment of cancer, autoimmune diseases, ectopic pregnancy, and for the induction of medical abortions. It acts by inhibiting the metabolism of folic acid. For cancer, methotrexate competitively inhibits dihydrofolate reductase (DHFR), an enzyme that participates in the tetrahydrofolate synthesis. The affinity of methotrexate for DHFR is about one thousand-fold that of folate. DHFR catalyses the conversion of dihydrofolate to the active tetrahydrofolate. Folic acid is needed for the de novo synthesis of the nucleoside thymidine, required for DNA synthesis. Also, folate is essential for purine and pyrimidine base biosynthesis, so synthesis will be inhibited. Methotrexate, therefore, inhibits the synthesis of DNA, RNA, thymidylates, and proteins.

Pemetrexed (brand name Alimta®) is a chemotherapy drug manufactured and marketed by Eli Lilly and Company. Pemetrexed is chemically similar to folic acid and is in the class of chemotherapy drugs called folate antimetabolites. It works by inhibiting three enzymes used in purine and pyrimidine synthesis—thymidylate synthase (TS), dihydrofolate reductase (DHFR), and glycinamide ribonucleotide formyltransferase (GARFT). By inhibiting the formation of precursor purine and pyrimidine nucleotides, pemetrexed prevents the formation of DNA and RNA, which are required for the growth and survival of both normal cells and cancer cells.

Examples of other anti-folates include trimetrexate, edatrexate, lometrexol, 5-fluorouracil, pralatrexate, aminopterin, proguanil, pyrimethamine, and trimethoprin.

### II. A2b ADENOSINE RECEPTOR ANTAGONISTS

The biological effects of adenosine are mediated by G protein-coupled plasma membrane receptors. Four adenosine receptor subtypes have been demonstrated: A1 adenosine receptor (A1R), A2a adenosine receptor (A2AR), A2b adenosine receptor (A2BR), and A3 adenosine receptor (A3R). Of the four adenosine receptors, the A2b receptor has the weakest affinity for adenosine. For this reason, it is not activated under physiological normal conditions in contrast to the other adenosine receptors. The A1 and A3 receptors are coupled to Gi proteins and inhibit adenylate cyclase, while the A2a and A2b receptors stimulate adenylate cyclase thereby causing an intracellular increase in cAMP Gs proteins.

A2BRs are expressed on pulmonary epithelial cells, vascular endothelial cells, smooth muscle cells, fibroblasts, and inflammatory cells. The expression of the A2BR on the cell surface is a dynamic process and is, for example, greatly increased by hypoxia, inflammatory factors, and free radicals. The activation of A2BR leads to the formation and release of pro-inflammatory and pro-fibrotic cytokines, such as IL-6, IL-4 and IL-8. In tumors and tumor-surrounding tissues, the local adenosine concentration is frequently elevated. This leads to activation of the above-described adenosine receptors on tumor cells, tumor-associated tumor cells, and cells of the surrounding tissue. The resulting initiated signaling pathways trigger various processes that promote tumor growth and spread to other places in the body.

A2BR antagonists are well known and have been described in numerous patents and patent publications. For example, A2BR antagonists may be selected from the group consisting of MRS1706, MRS1754, PSB603, and CVT-6883. In addition, the following patents and patent publications are representative examples that provide A2BR antagonists, all of which are incorporated herein by reference: U.S. Pat. Nos. 4,548,820; 5,734,051; 5,734,052; 6,117,878; 6,180,791; 6,545,002; 6,806,270; 6,825,349; 6,977,300; 7,125,993; 7,189,717; 7,205,403; 7,342,006; 7,579,348; 7,601,723; 7,618,962; U.S. Pat. Appln. Publn. No. 2003/0229067; PCT Pat. Appln. Publn. Nos. WO2006/028810; WO2003063800; WO2000/73307; WO2003/002566; WO2003/006465; WO2003/042214; WO2003/053366; WO2003/063800; WO1994/26744; WO2001/16134; and European Pat. Nos. EP0698607; EP0619316; EP0607607; EP0590919; EP0559893; EP0449175; EP0389282; EP0267607; EP0203721; and, EP0092398.

### III. IMMUNE CHECKPOINT INHIBITORS

Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), programmed death-ligand 1 (PD-L1), T-cell immunoglobulin domain and mucin domain 3 (TIM-3), and V-domain Ig suppressor of T cell activation (VISTA). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of Nivolumab (also known as MDX-1106-04, MDX-1106, MK-347, ONO-4538, BMS-936558, and OPDIVO®; described in WO2006/121168), Pembrolizumab (also known as MK-3475, Merck 3475, lambrolizumab, KEYTRUDA®, and SCH-900475; WO2009/114335), Pidilizumab (also known as CT-011, hBAT or hBAT-1; WO2009/101611), Cemiplimab (also known as LIBTAYO®, REGN-2810, REGN2810, SAR-439684, SAR439684), Tislelizumab (also known as BGB-A317, hu317-1/IgG4mt2; U.S. Pat. No. 8,735,553), Spartalizumab (also known as PDR001, PDR-001, NPV-PDR001, NPVPDR001; U.S. Pat. No. 9,683,048), PF-06801591, AK105, BCD-100, BI-754091, HLX10, JS001, LZMO09, MEDI 0680, MGA012, Sym021, TSR-042, MGD013, AK104 (bispecific with anti-CTLA4), and XmAb20717 (bispecific with anti-CTLA4).

In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). For example, AMP-224 (also known as B7-DCIg) is a PD-L2-Fc fusion soluble receptor described in WO2010/027827 and WO2011/066342.

In some embodiment, a PD-L1 binding antagonist is an anti-PD-L1 antibody (e.g., a human antibody, a humanized antibody, or a chimeric antibody). In some embodiments, the anti-PD-L1 antibody is selected from the group consisting of Atezolizumab (also known as Tencentriq, MPDL3280A; described in U.S. Pat. No. 8,217,149), Avelumab (also known as BAVENCIO®, MSB-0010718C, MSB0010718C), Durvalumab (also known as IMFINZI®, MEDI-4736, MEDI4736; described in WO2011/066389), FS118, BCD-135, BGB-A333, CBT502 (also known as TQB2450), CK-301, CS1001 (also known as WBP3155), FAZ053, KN035, MDX-1105, MSB2311, SHR-1316, M7824, LY3415244, CA-170, and CX-072.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, MDX-CTLA4, and YERVOY®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab).

In some embodiment, a CTLA-4 binding antagonist is an anti-CTLA-4 antibody (e.g., a human antibody, a humanized antibody, or a chimeric antibody). In some embodiments, the anti-CTLA-4 antibody is selected from the group consisting of ipilimumab (also known as 10D1, MDX-010, MDX-101, MDX-CTLA4, and YERVOY®; described in WO 01/14424), Tremelimumab (also known as CP-675,206, CP-675, ticilimumab; described in WO 00/37504), BMS-986218, AK104 (bispecific with anti-PD-1), and XmAb20717 (bispecific with anti-PD-1).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### IV. METHODS OF TREATMENT

The term “subject” or “patient” as used herein refers to any individual to which the subject methods are performed. Generally the patient is human, although as will be appreciated by those in the art, the patient may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The methods described herein are useful in inhibiting survival or proliferation of cells (e.g., tumor cells), treating proliferative disease (e.g., cancer, psoriasis), and treating pathogenic infection. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, previous therapy, the patient's clinical history and response to the agent, and the discretion of the physician. The agent may be suitably administered to the patient at one time or over a series of treatments.

### V. COMBINATION TREATMENTS

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a combination of an antifolate agent and an A2BR antagonist is “A” and another anti-cancer therapy (e.g., immunotherapy) is “B”:

| | | | | | | | |
|---|---|---|---|---|---|---|---|
| A/B/A | B/A/B | B/B/A | A/A/B | A/B/B | B/A/A | A/B/B/B | B/A/B/B |
| B/B/B/A | B/B/A/B | A/A/B/B | A/B/A/B | A/B/B/A | B/B/A/A | | |
| B/A/B/A | B/A/A/B | A/A/A/B | B/A/A/A | A/B/A/A | A/A/B/A | | |

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311). It is contemplated that one or more anti-cancer therapies may be employed with the antibody therapies described herein.

In some embodiment, the immune therapy could be adoptive immunotherapy, which involves the transfer of autologous antigen- specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs'surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### VI. KITS

In various aspects of the invention, a kit is envisioned containing, diagnostic agents, therapeutic agents and/or delivery agents. In some embodiments, the present invention contemplates a kit for preparing and/or administering a therapy of the invention. The kit may comprise reagents capable of use in administering an active or effective agent(s) of the invention. Reagents of the kit may include one or more anti-cancer component of a combination therapy, as well as reagents to prepare, formulate, and/or administer the components of the invention or perform one or more steps of the inventive methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods, and will follow substantially the same procedures as described herein or are known to those of ordinary skill.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—MTAP-Deficient Bladder Cancer is Associated with Poor Clinical Outcome and Correlates with Sensitivity to Pemetrexed

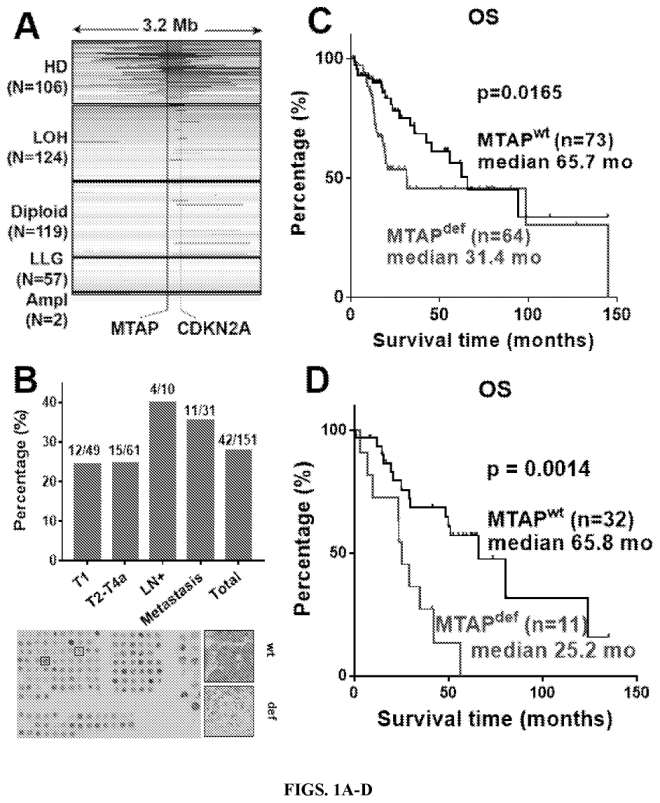

FIG. 1A illustrates the MTAP homozygous deletion (HD) rate in TCGA. FIG. 1B illustrates the MTAP protein deficiency rate in bladder cancer based on immunohistochemistry of a tissue microarray. FIG. 1C illustrates the median overall survival of MTAP-wild type vs. MTAP-deficient bladder cancer patients in the TCGA database. FIG. 1D illustrates that median overall survival of MTAP-wild type vs. MTAP-deficient bladder cancer patients in the MD Anderson database.

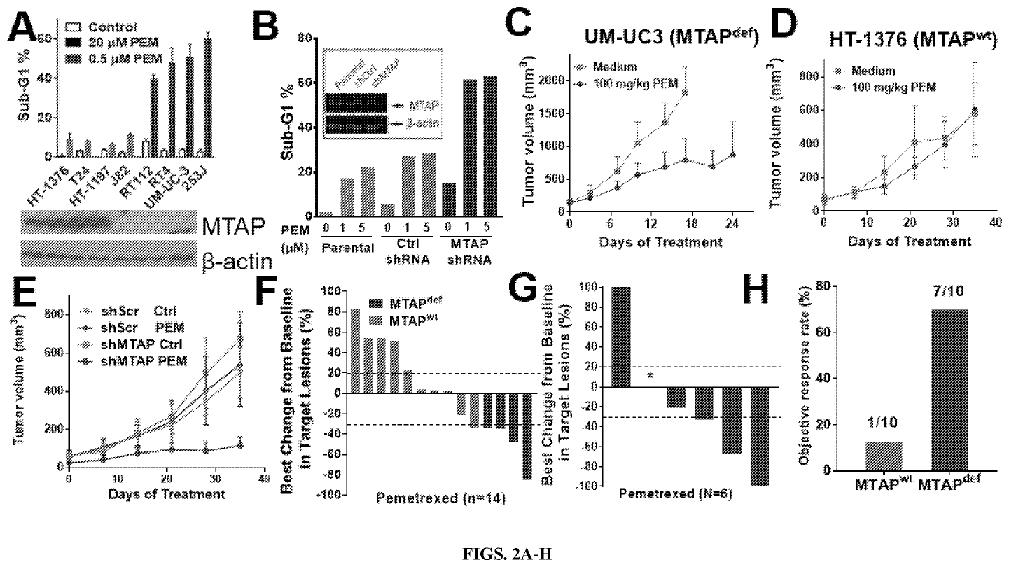

Pemetrexed was at least 40-fold more potent in inducing apoptosis (as detected by sub-G1 population) in MTAP-deficient vs. MTAP-wild type human bladder cancer cell lines (FIG. 2A). In addition, MTAP knockdown in human bladder cell lines resulted in significant apoptosis with pemetrexed treatment (FIG. 2B). Pemetrexed inhibited MTAP-deficient human tumor growth (FIG. 2C) but not MTAP-wild type tumor growth (FIG. 2D) in a xenograft mouse model. Pemetrexed also inhibited growth of MTAP-knockdown human tumors (FIG. 2E). Finally, pemetrexed had a higher response rate in patients with MTAP-deficient bladder cancer vs MTAP-wild type bladder cancer in retrospective (FIG. 2F), prospective (FIG. 2G), and combined retrospective and prospective data (FIG. 2H).

### Example 2—MTAP-Deficient Bladder Cancer is Associated with Sensitivity to Pemetrexed in Combination with anti-PD-L1

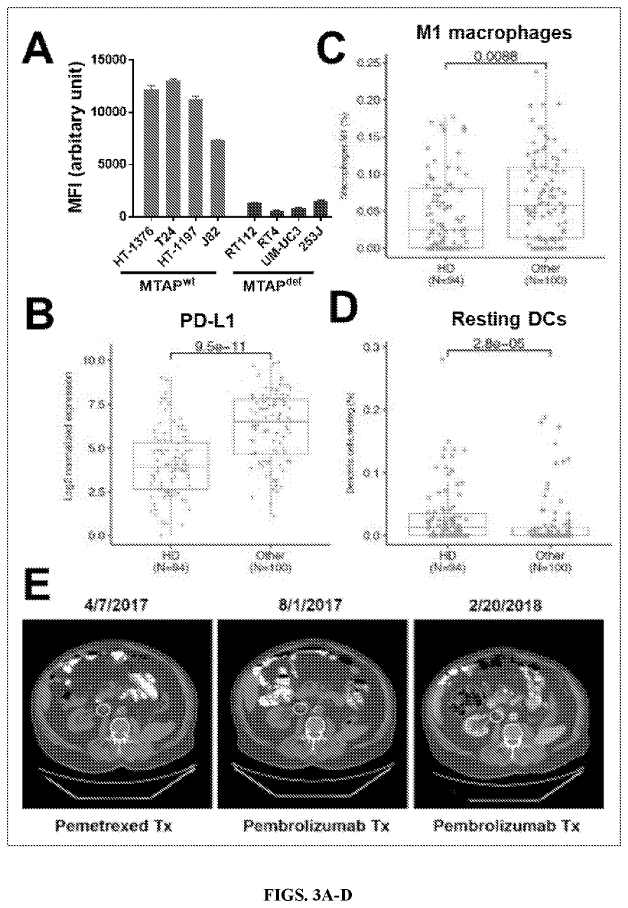

MTAP-deficient bladder tumors have a “cold” immune microenvironment. For example, MTAP-deficient human bladder cancer cell lines manifest significantly lower expression levels of PD-L1 compared to MTAP-proficient bladder cancer cell lines (FIG. 3A). Analysis of TCGA gene expression data further indicated that MTAP-deficient tumors have a lower M1 macrophage signature (FIG. 3B), lower PD-L1 (CD274) expression (FIG. 3C), and higher resting dendritic cell signature (FIG. 3D). An analysis of retrospective data revealed that 1 of 6 patients with metastatic MTAP-deficient bladder cancer responded to anti-PD1/PD-L1 therapy and this patient was pre-treated with pemetrexed (FIG. 3E).

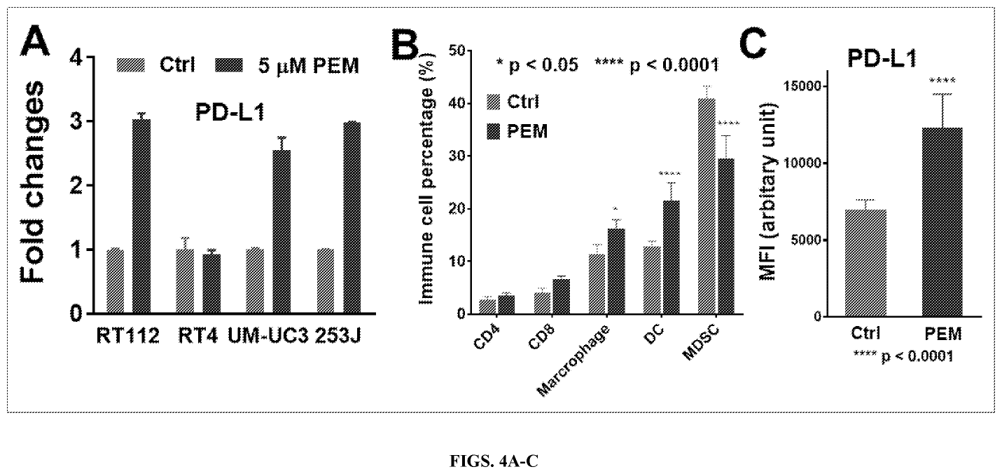

However, pemetrexed treatment resulted in a “hot” immune microenvironment. For example, pemetrexed induced PD-L1 in MTAP-deficient bladder cancer cell lines (FIG. 4A). Pemetrexed also increased CD4/8 T cells, macrophages, and dendritic cells and decreased MDSCs in vivo on MB49 tumors (FIG. 4B) In addition, pemetrexed induced PD-L1 in vivo on MB49 tumors (FIG. 4C). Based on this, FIG. 6 provides a proposed phase 2 trial schema for combination treatment with pemetrexed and an anti-PD-L1 therapy (e.g., avelumab).

### Example 3—MTAP-Deficient Bladder Cancer is Associated with Sensitivity to Pemetrexed in Combination with A2BR Antagonists

MTAP-deficient tumors may be resistant to immuno-checkpoint therapy but particularly sensitive to pemetrexed. MTAP deficiency leads to accumulation of its substrate MTA, which binds receptor A2BR on T cells to suppress T cell function, IFN signaling, and PD-L1 expression. Pemetrexed inhibits de novo adenosine synthesis and is highly cytotoxic to MTAP-deficient tumor which lacks salvage adenosine synthesis. Pemetrexed also increases tumor PD-L1 expression.

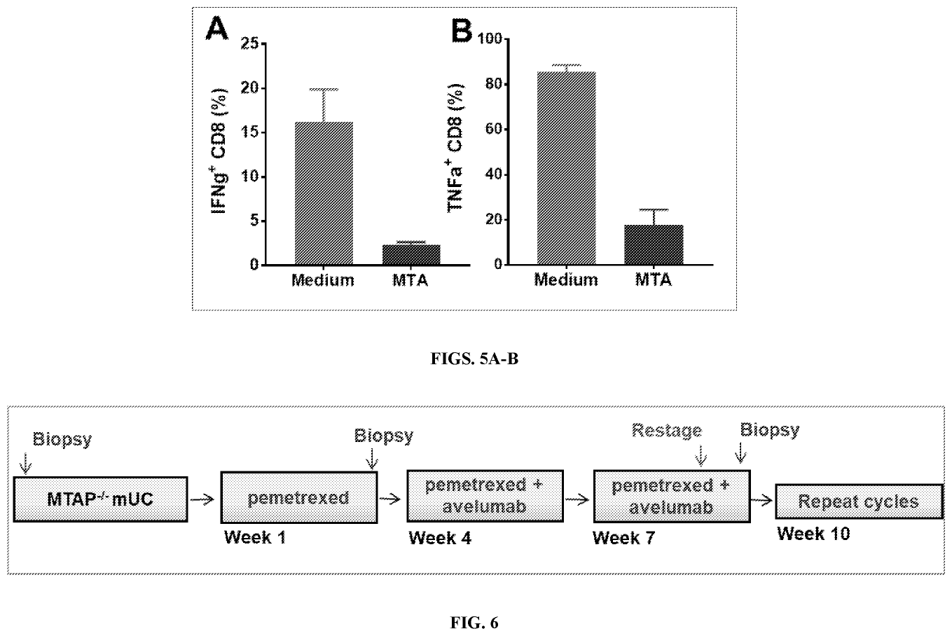

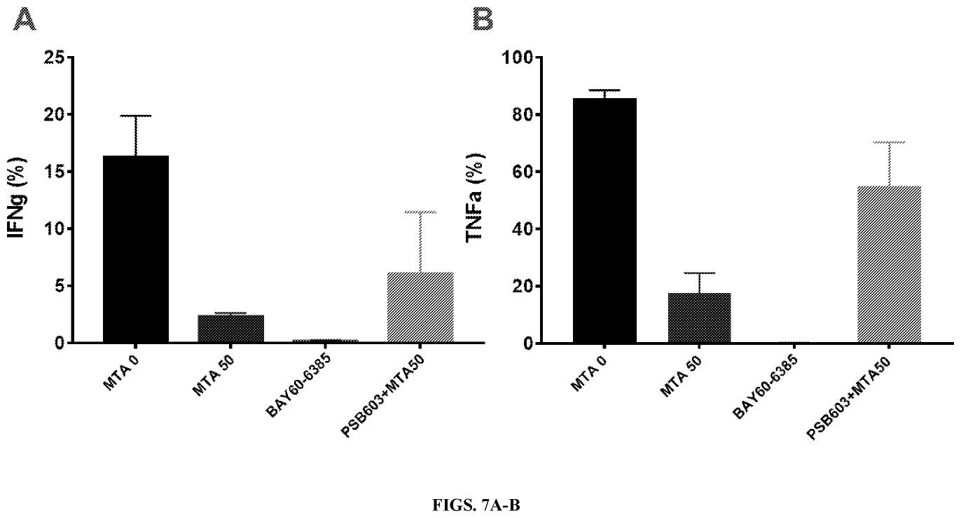

In order to demonstrate the effect of MTA on T cell function, mouse CD8 T cells were isolated from spleen and activated with anti-CD³/anti-CD28 in vitro. MTA was added into culture for 72 h. After incubation, the data show that MTA was able to significantly inhibit production of INF-γ (FIGS. 5A & 7A) and tumor necrosis factor TNF-α (FIGS. 5B & 7B). A specific agonist to the MTA receptor A2BR (Bay60-6385) was able to mimic this inhibition on T cell function (FIGS. 7A & 7B), while the A2BR antagonist (PSB-603) was able to partially rescue MTA-induced inhibition on the production of INF-γ and TNF-α (FIGS. 7A & 7B).

### Example 4—MTAP-Deficient Bladder Cancer is Associated with Sensitivity to Pemetrexed in Combination with A2BR Antagonists and Anti-PD-L1 (or anti-PD1)

MTAP-deficient tumors may be resistant to immuno-checkpoint therapy but particularly sensitive to pemetrexed. MTAP deficiency leads to accumulation of its substrate MTA, which binds receptor A2BR on T cells to suppress T cell function, IFN signaling, and PD-L1 expression. Pemetrexed inhibits de novo adenosine synthesis and is highly cytotoxic to MTAP-deficient tumors, which lack salvage adenosine synthesis. Pemetrexed also increases tumor PD-L1 expression.

In order to demonstrate the effect of MTA on T cell function, mouse CD8 T cells were isolated from spleen and activated with anti-CD³/anti-CD28 in vitro. MTA was added into culture for 72 h. After incubation, the data show that MTA was able to significantly inhibit production of INF-γ (FIGS. 5A & 7A) and tumor necrosis factor TNF-α (FIGS. 5B & 7B). A specific agonist to the MTA receptor A2BR (Bay60-6385) was able to mimic this inhibition on T cell function (FIGS. 7A & 7B), while the A2BR antagonist (PSB-603) was able to partially rescue MTA-induced inhibition on the production of INF-γ and TNF-α (FIGS. 7A & 7B). Therefore, a triple combination therapy with pemetrexed +A2BR antagonists+anti-PD-L1 (or anti-PD1) can be ultra-effective for MTAP-deficient malignancies through five different mechanisms: (1) direct cytotoxicity (from pemetrexed); (2) decrease of MTA levels (from pemetrexed cytotoxicity) and thus alleviation of inhibition of IFN signaling and T cell function; (3) increases in levels of tumor PD-L1 expression (from pemetrexed); (4) T cell activation by mitigating MTA inhibition (A2BR antagonists); and (5) further T cell activation by inhibiting checkpoint molecule PD-L1 (anti-PD-L1 or anti-PD1).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Paz-Ares et al. Cancer 97:2056; 2003
Sweeney et al. J Clin Oncol 24:3451; 2006
Galsky et al. Invest New Drugs 25:265; 2007

What is claimed is:

1. A method of treating a patient having a cancer, the method comprising (a) determining or having determined the level of MTAP protein expression in the patient's cancer; (b) selecting or having selected the patient for treatment with an anti-folate agent and an A2BR antagonist when the patient's cancer has a decreased level of MTAP expression relative to a level of MTAP protein expression in a reference sample; and (c) administering or having administered to the selected patient a combined therapeutically effective amount of an anti-folate agent and an A2BR antagonist.

2. A method of treating a patient having a cancer, the method comprising administering to the patient a combined therapeutically effective amount of an anti-folate agent and an A2BR antagonist, wherein the cancer has a decreased level of MTAP expression relative to a level of MTAP protein expression in a reference sample.

3. The method of claim 1 or 2, wherein the anti-folate agent comprises pemetrexed, methotrexate, trimetrexate, edatrexate, lometrexol, 5-fluorouracil, pralatrexate, aminopterin, proguanil, pyrimethamine, or trimethoprin.

4. The method of any one of claims 1-3, wherein the A2BR antagonist comprises MRS1706, MRS1754, PSB603, CVT-6883, or PBF-1129, or an adenosine 2A (A2A) receptor antagonist with overlapping inhibiting activity of A2BR.

5. The method of any one of claims 1-4, further comprising administering to the patient an immune checkpoint inhibitor.

6. The method of claim 5, wherein the immune checkpoint inhibitor comprises one or more of an anti-PD1 therapy, an anti-PD-L1 therapy, and an anti-CTLA-4 therapy.

7. The method of claim 6, wherein the anti-PD1 therapy comprises nivolumab, pembrolizumab, pidilizumab, cemiplimab, tislelizumab, spartalizumab, PF-06801591, AK105, BCD-100, BI-754091, HLX10, JS001, LZMO09, MEDI 0680, MGA012, Sym021, TSR-042, MGD013, AK104, and/or XmAb20717.

8. The method of claim 6, wherein the anti-PD-L1 therapy comprises atezolizumab, avelumab, durvalumab, FS118, BCD-135, BGB-A333, CBT502, CK-301, CS1001, FAZ053, KN035, MDX-1105, MSB2311, SHR-1316, M7824, LY3415244, CA-170, and CX-072.

9. The method of claim 6, wherein the anti-CTLA-4 therapy comprises ipilimumab, tremelimumab, BMS-986218, AK104, and/or XmAb20717.

10. The method of any one of claims 1-9, wherein step (a) comprises (i) obtaining or having obtained a biological sample from the patient; and (ii) performing or having performed an assay on the biological sample to determine the level of MTAP protein expression in the patient's cancer.

11. The method of any one of claims 1-10, wherein the reference sample is obtained from healthy or non-cancerous tissue in the patient.

12. The method of any one of claims 1-10, wherein the reference sample is obtained from a healthy subject.

13. The method of any one of claims 1-12, wherein the cancer has an MTAP deletion.

14. The method of any one of claims 1-13, further comprising administering a further anti-cancer therapy to the patient.

15. The method of claim 14, wherein the further anti-cancer therapy is a surgical therapy, a chemotherapy, a radiation therapy, a cryotherapy, a hormonal therapy, a toxin therapy, an immunotherapy, or a cytokine therapy.

16. The method of any one of claims 1-15, wherein the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

17. The method of any one of claims 1-15, wherein the cancer is a bladder cancer, a lung cancer, a mesothelioma, a glioblastoma, or a lymphoma.

18. The method of any one of claims 1-17, wherein the patient has previously undergone at least one round of anti-cancer therapy.

19. The method of any one of claims 1-18, further comprising reporting the level of MTAP protein expression in the patient's cancer.

20. The method of claim 19, wherein reporting comprises preparing a written or electronic report.

21. The method of claim 19 or 20, further comprising providing the report to the subject, a doctor, a hospital, or an insurance company.

22. The method of any one of claims 1-21, wherein the patient has previously failed to respond to the administration of an immune checkpoint inhibitor.

23. The method of any one of claims 1-22, wherein the method is a method of overcoming resistance to immune checkpoint inhibitor therapy.

24. The method of any one of claims 1-23, wherein the method is further defined as a method for increasing sensitivity to immunotherapy.

25. A method of selecting a patient having a cancer for treatment with an anti-folate agent and an A2BR antagonist, the method comprising (a) determining or having determined the level of MTAP protein expression in the patient's cancer; and (b) selecting or having selected the patient for treatment with an anti-folate agent and an A2BR antagonist when the patient's cancer has a decreased level of MTAP expression relative to a level of MTAP protein expression in a reference sample.

26. The method of claim 25, wherein step (a) comprises (i) obtaining or having obtained a biological sample from the patient; and (ii) performing or having performed an assay on the biological sample to determine the level of MTAP protein expression in the patient's cancer.

27. The method of claim 25 or 26, wherein the reference sample is obtained from healthy or non-cancerous tissue in the patient.

28. The method of claim 25 or 26, wherein the reference sample is obtained from a healthy subject.

29. The method of any one of claims 25-28, wherein the cancer has an MTAP deletion.

30. The method of any one of claims 25-29, further comprising (c) administering or having administered to the selected patient combined therapeutically effective amounts of an anti-folate agent and an A2BR antagonist.

31. The method of any one of claims 25-30, wherein the anti-folate agent comprises pemetrexed, methotrexate, trimetrexate, edatrexate, lometrexol, 5-fluorouracil, pralatrexate, aminopterin, proguanil, pyrimethamine, or trimethoprin.

32. The method of any one of claims 25-31, wherein the A2BR antagonist comprises MRS1706, MRS1754, PSB603, CVT-6883, or PBF-1129, or an adenosine 2A (A2A) receptor antagonist with overlapping inhibiting activity of A2BR.

33. The method of any one of claims 30-32, further comprising administering to the patient an immune checkpoint inhibitor.

34. The method of claim 33, wherein the immune checkpoint inhibitor comprises one or more of an anti-PD1 therapy, an anti-PD-L1 therapy, and an anti-CTLA-4 therapy.

35. The method of claim 34, wherein the anti-PD1 therapy comprises nivolumab, pembrolizumab, pidilizumab, cemiplimab, tislelizumab, spartalizumab, PF-06801591, AK105, BCD-100, BI-754091, HLX10, JS001, LZMO09, MEDI 0680, MGA012, Sym021, TSR-042, MGD013, AK104, and/or XmAb20717.

36. The method of claim 34, wherein the anti-PD-L1 therapy comprises atezolizumab, avelumab, durvalumab, FS118, BCD-135, BGB-A333, CBT502, CK-301, CS1001, FAZ053, KN035, MDX-1105, MSB2311, SHR-1316, M7824, LY3415244, CA-170, and CX-072.

37. The method of claim 34, wherein the anti-CTLA-4 therapy comprises ipilimumab, tremelimumab, BMS-986218, AK104, and/or XmAb20717.

38. The method of any one of claims 25-37, wherein the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

39. The method of any one of claims 25-37, wherein the cancer is a bladder cancer, a lung cancer, a mesothelioma, a glioblastoma, or a lymphoma.

40. The method of any one of claims 25-39, wherein the patient has previously undergone at least one round of anti-cancer therapy.

41. The method of any one of claims 25-40, further comprising reporting the level of MTAP protein expression in the patient's cancer.

42. The method of claim 41, wherein reporting comprises preparing a written or electronic report.

43. The method of claim 41 or 42, further comprising providing the report to the subject, a doctor, a hospital, or an insurance company.

44. A method of treating a patient having a cancer, the method comprising (a) determining or having determined the level of MTAP protein expression in the patient's cancer; (b) selecting or having selected the patient for treatment with an anti-folate agent and an immune checkpoint inhibitor when the patient's cancer has a decreased level of MTAP expression relative to a level of MTAP protein expression in a reference sample; and (c) administering or having administered to the selected patient a combined therapeutically effective amount of an anti-folate agent and an immune checkpoint inhibitor.

45. A method of treating a patient having a cancer, the method comprising administering to the patient a combined therapeutically effective amount of an anti-folate agent and an immune checkpoint inhibitor, wherein the cancer has a decreased level of MTAP expression relative to a level of MTAP protein expression in a reference sample.

46. The method of claim 44 or 45, wherein the anti-folate agent comprises pemetrexed, methotrexate, trimetrexate, edatrexate, lometrexol, 5-fluorouracil, pralatrexate, aminopterin, proguanil, pyrimethamine, or trimethoprin.

47. The method of any one of claims 44-46, wherein the immune checkpoint inhibitor comprises one or more of an anti-PD1 therapy, an anti-PD-L1 therapy, and an anti-CTLA-4 therapy.

48. The method of claim 47, wherein the anti-PD1 therapy comprises nivolumab, pembrolizumab, pidilizumab, cemiplimab, tislelizumab, spartalizumab, PF-06801591, AK105, BCD-100, BI-754091, HLX10, JS001, LZMO09, MEDI 0680, MGA012, Sym021, TSR-042, MGD013, AK104, and/or XmAb20717.

49. The method of claim 47, wherein the anti-PD-L1 therapy comprises atezolizumab, avelumab, durvalumab, FS118, BCD-135, BGB-A333, CBT502, CK-301, CS1001, FAZ053, KN035, MDX-1105, MSB2311, SHR-1316, M7824, LY3415244, CA-170, and CX-072.

50. The method of claim 47, wherein the anti-CTLA-4 therapy comprises ipilimumab, tremelimumab, BMS-986218, AK104, and/or XmAb20717.

51. The method of any one of claims 44-50, further comprising administering to the patient an A2BR antagonist.

52. The method of claim 51, wherein the A2BR antagonist comprises MRS1706, MRS1754, PSB603, CVT-6883, or PBF-1129, or an adenosine 2A (A2A) receptor antagonist with overlapping inhibiting activity of A2BR.

53. The method of any one of claims 44-52, wherein step (a) comprises (i) obtaining or having obtained a biological sample from the patient; and (ii) performing or having performed an assay on the biological sample to determine the level of MTAP protein expression in the patient's cancer.

54. The method of any one of claims 44-53, wherein the reference sample is obtained from healthy or non-cancerous tissue in the patient.

55. The method of any one of claims 44-53, wherein the reference sample is obtained from a healthy subject.

56. The method of any one of claims 44-55, wherein the cancer has an MTAP deletion.

57. The method of any one of claims 44-56, further comprising administering a further anti-cancer therapy to the patient.

58. The method of claim 57, wherein the further anti-cancer therapy is a surgical therapy, a chemotherapy, a radiation therapy, a cryotherapy, a hormonal therapy, a toxin therapy, an immunotherapy, or a cytokine therapy.

59. The method of any one of claims 44-58, wherein the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

60. The method of any one of claims 44-58, wherein the cancer is a bladder cancer, a lung cancer, a mesothelioma, a glioblastoma, or a lymphoma.

61. The method of any one of claims 44-60, wherein the patient has previously undergone at least one round of anti-cancer therapy.

62. The method of any one of claims 44-61, further comprising reporting the level of MTAP protein expression in the patient's cancer.

63. The method of claim 62, wherein reporting comprises preparing a written or electronic report.

64. The method of claim 62 or 63, further comprising providing the report to the subject, a doctor, a hospital, or an insurance company.

65. The method of any one of claims 44-64, wherein the patient has previously failed to respond to the administration of an immune checkpoint inhibitor.

66. The method of any one of claims 44-65, wherein the method is a method of overcoming resistance to immune checkpoint inhibitor therapy.

67. The method of any one of claims 44-66, wherein the method is further defined as a method for increasing sensitivity to immunotherapy.

68. A method of selecting a patient having a cancer for treatment with an anti-folate agent and an immune checkpoint inhibitor, the method comprising (a) determining or having determined the level of MTAP protein expression in the patient's cancer; and (b) selecting or having selected the patient for treatment with an anti-folate agent and an immune checkpoint inhibitor when the patient's cancer has a decreased level of MTAP expression relative to a level of MTAP protein expression in a reference sample.

69. The method of claim 68, wherein step (a) comprises (i) obtaining or having obtained a biological sample from the patient; and (ii) performing or having performed an assay on the biological sample to determine the level of MTAP protein expression in the patient's cancer.

70. The method of claim 68 or 69, wherein the reference sample is obtained from healthy or non-cancerous tissue in the patient.

71. The method of claim 68 or 69, wherein the reference sample is obtained from a healthy subject.

72. The method of any one of claims 68-71, wherein the cancer has an MTAP deletion.

73. The method of any one of claims 68-72, further comprising (c) administering or having administered to the selected patient combined therapeutically effective amounts of an anti-folate agent and an immune checkpoint inhibitor.

74. The method of claim any one of claims 68-73, wherein the anti-folate agent comprises pemetrexed, methotrexate, trimetrexate, edatrexate, lometrexol, 5-fluorouracil, pralatrexate, aminopterin, proguanil, pyrimethamine, or trimethoprin.

75. The method of any one of claims 68-74, wherein the immune checkpoint inhibitor comprises one or more of an anti-PD1 therapy, an anti-PD-L1 therapy, and an anti-CTLA-4 therapy.

76. The method of claim 75, wherein the anti-PD1 therapy comprises nivolumab, pembrolizumab, pidilizumab, cemiplimab, tislelizumab, spartalizumab, PF-06801591, AK105, BCD-100, BI-754091, HLX10, JS001, LZMO09, MEDI 0680, MGA012, Sym021, TSR-042, MGD013, AK104, and/or XmAb20717.

77. The method of claim 75, wherein the anti-PD-L1 therapy comprises atezolizumab, avelumab, durvalumab, FS118, BCD-135, BGB-A333, CBT502, CK-301, CS1001, FAZ053, KN035, MDX-1105, MSB2311, SHR-1316, M7824, LY3415244, CA-170, and CX-072.

78. The method of claim 75, wherein the anti-CTLA-4 therapy comprises ipilimumab, tremelimumab, BMS-986218, AK104, and/or XmAb20717.

79. The method of any one of claims 73-78, further comprising administering to the patient an A2BR antagonist.

80. The method of claim 79, wherein the A2BR antagonist comprises MRS1706, MRS1754, PSB603, CVT-6883, or PBF-1129, or an adenosine 2A (A2A) receptor antagonist with overlapping inhibiting activity of A2BR.

81. The method of any one of claims 68-80, wherein the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

82. The method of any one of claims 68-80, wherein the cancer is a bladder cancer, a lung cancer, a mesothelioma, a glioblastoma, or a lymphoma.

83. The method of any one of claims 68-82, wherein the patient has previously undergone at least one round of anti-cancer therapy.

84. The method of any one of claims 68-83, further comprising reporting the level of MTAP protein expression in the patient's cancer.

85. The method of claim 84, wherein reporting comprises preparing a written or electronic report.

86. The method of claim 84 or 85, further comprising providing the report to the subject, a doctor, a hospital, or an insurance company.
